(19) United States
(12) Patent Application Publication
(10) Pub. No.: US 2005/0123922 A1
(43) Pub. Date: Jun. 9, 2005

(54) Method for the selection of compounds useful for the treatment of huntington's disease

(76) Inventors: Elena Cattaneo, Brugherio (IT); Chiara Zuccato, Cassano Magnago (IT)

Correspondence Address: WILLIAM COLLARD;COLLARD & ROE, P.C., 1077 NORTHERN BOULEVARD, ROSLYN, NY, 11576, US

(21) Appl. No.: 10/511,665

(22) PCT Filed: Apr. 17, 2003
(86) PCT No.: PCT/IB03/01627

(30) Foreign Application Priority Data
Apr. 17, 2002 (IT) MI2002A000809

(51) Int. Cl. C12Q 1/68; C07H 21/04; C12P 21/04; C07K 14/705; C07K 14/435

(52) U.S. Cl. 435/6; 435/69.7; 435/320.1; 435/325; 530/350; 536/23.5

(57) ABSTRACT

The present invention describes a screening method for the identification of molecules useful in the prevention and/or treatment of Huntington's disease characterized by the evaluation of the ability of said molecules of inhibiting the activity of the NRSE sequence; the method is performed by incubating said molecules in a cell system appropriately engineered to detect the level of activation of the NRSE sequence. The invention also describes the process for the preparation of said cell system, the vector used in said process and the use of molecules inhibiting, the activity of the NRSE sequence for the preparation of medicaments useful in the treatment and/or prevention of Huntington's disease.

### FIELD OF THE INVENTION

This invention relates to the field of central nervous system diseases therapy and in particular to the field of drug-screening system for the isolation of compounds useful for the treatment of Huntington's Disease.

### BACKGROUND

Huntington's Disease (HD) is an inherited autosomal dominant disorder starting in midlife and with variable penetrance. The neurological damage comprises the selective neuronal loss of the striatal neurons in the basal ganglia, a cerebral region which is central of involuntary movements. Huntington's Disease progression is slow and inexorable during which patients lose motor and cognitive ability and acquire psychiatric disturbance (Hayden MR Huntington's chorea, Springer-Verlag:London, Berlin Heidelberg. 1981).

Usually, the disease onset is in fertile age (35 years of age) with an estimate prevalence of 1 out of 10000 individuals and an average duration of about 17 years until death.

Frequently, Huntington patients become aware of their situation and future even before any manifested behavioural alteration and thus progressively isolate themselves from society, quit their job or social activity. In addition to the consequences for the patients and their families, the long clinical course of the disease results am high economic cost for the society.

The genetic defect responsible for the pathology was identified in 1993 and it is based on a mutation within the gene encoding a protein called huntingtin which is beneficial for brain neurons.

The mutation of the gene consists in an aberrant expansion of a CAG triplet nucleotides located at the beginning of the gene which produces, in the encoded protein, the aminoacid glutamine.

In the disease state, the amino-terminal portion of huntingtin therefore contains a polyglutamine expansion which causes the production of mutant huntingtin protein with a newly acquired toxic function (gain of function mechanism). In addition to that, the mutation also causes the loss of the neuronal protective function of the wild-type protein (loss of function mechanism).

The loss of wild-type huntingtin function together with the new toxic activity acquired by the mutant protein cause the progressive degeneration of brain striatal neurons which control the involuntary movements, leading to choreic manifestations.

Recently, it has been found that, in physiologic conditions, normal huntingtin is able to stimulate the production of a neurotrophin called BDNF (Brain-Derived Neurotrophic Factor), (Zuccato, Science 2001, 293, pp 493-8), a growth factor which is part of the NGF (Nerve Growth Factor) family of neuronal growth factor, which is a pro-survival factor for the neurons in the central nervous system, and in particular for the striatal neurons which are the most affected in the pathology.

In particular, it has been previously discovered that wild-type huntingtin stimulates the production of BDNF by increasing the transcription of the gene encoding for this protein. This function is lost in the pathology, and as a consequence, availabilty of BDNF to the striatal neurons is highly reduced (Zuccato, Science 2001, see above).

The structure of the gene responsable for BDNF production is known (and represented in a schematic way in FIG. 1). On the contrary, much less is known about the mechanism of transcription of this gene. In particular, there are no studies showing the presence of a sequence within the BDNF gene able to function as a target for wild-type huntingtin function.

At the moment there are no specific therapies available which can prevent symptoms or cure patients affected with Huntington's Disease.

The choreic movements typical of the disease can be partially reduced with antipsychotics and reserpine (Merck Manual, Ed. Merck Res. Laboratories, 17^{th} ed., 1999, 1464); it is therefore clear the interest in the development of pharmacological approaches able to delay the disease onset, to reduce the severity of the pathology and/or to slow its progression.

### SUMMARY

The present invention is based on the finding made by the applicants, of the molecular target of wild-type huntingtin, represented by the smallest DNA sequence which is necessary for huntingtin to execute its action on BDNF gene transcription and thus to exert its neuroprotective action.

This target sequence is a palindromic sequence of 53 basepairs (5′-TCCATTCAGCACCTTGGACAGAGCCAGCGGATTTGTCCGAGGTGGTAGA CTT-3′) which is located within the BDNF gene promoter region II. Such a sequence which is called NRSE (Neuron Restrictive Silencer Element) is well known and it is present in various genes.

The applicants have now found that this sequence within the BDNF gene acts as a molecular target for wild-type huntingtin activity, regulating therefore its neurotrophic and neuronal protective function. More specifically, the NRSE sequence has a silencer activity on the BDNF gene: wild-type huntingtin acting on this sequence blocks the silencer function which results wild type in the stimulation of the trascriptional activity of the BDNF gene. Even more specifically, the applicants have found that this block is due to the ability to sequester in the cytoplasm the main factor able to activate the NRSE sequence. This factor is called REST (Repressor Element RE1 Binding Transcription Factor) and it is a protein that, via its 9 structural zinc motives that recognize the DNA, is able to bind the NRSE sequence therefore silencing gene expression.

The invention makes use of these findings for the development of a drug screening system aiming at isolating molecules that, by being able to act as wild-type huntingtin, may be useful in the prevention or cure of Huntington's Disease.

The invention comprises a cellular system to carry out said drug screening method, a vector for the preparation of this cellular system and the use of molecules able to inhibit the NRSE sequence silencing activity with the final aim to identify drugs useful for the treatment of Huntington's Disease.

### DESCRIPTION OF FIGURES

FIG. 1 represents in a schematic way the structure of the BDNF gene with the grey region encoding for the protein and the promoter region upstream of the grey box. The promoter region is characterized by the presence of four sequences, each able to activate BDNF gene transcription independently and in a stimulus specific manner. These sequences are indicated in the figure with roman numbers I, II, III and IV. The NRSE silencer element is located upstream of the promoter sequence II. Number V indicates instead the portion of the gene which encodes the BDNF protein.

FIG. 2

Evaluation of CAT (chloramphenicol acetyle transferase) reporter gene activity in parental cells (P) and in cells engineered to overexpress wild-type or mutant huntingtin (FLwt and FLmu respectively, previously obtained in the laboratory, Rigamonti 2000), after transfection of BDNF II 1,1 CAT (panel A) and BDNF II 0,3 CAT (panel B) vectors. In particular, BDNF II 1,1 CAT construct contains a 1100 basepairs portion of promoter II, while the BDNF II 0,3 CAT construct contains a smaller part, composed of 300 basepairs of the same sequence. Both the sequences described contain the NRSE silencer element. In panels A and B, the arrows indicate are acetylated chloramphenicol. The result of the assay is an increased CAT activity in FLwt cells compared to control, which is represented by parental cells P which are not engineered. In the FLmu cells, in the presence of mutant huntingtin, CAT activity is dramatically reduced. This was verified, by the use of both vectors.

FIG. 3

Semiquantitative radioactive RT-PCR on seven genes (panel A to G) which are different from BDNF, but that are characterized by the presence of the NRSE sequence within their promoter. A=cholinacetyl transferase (ChAT); B=dynamin I; C=M₄ subunit of muscarinic receptor; D=B₂ subunit of nicotinic receptor; E=proenkephalin; F=synapsin I; G=VchAT.

The assay, performed in parental cells (P) or in cells engenereed to overexpress wild-type and mutant huntingtin (FLwt and FLmu), shows an increase in gene expression in the presence of the wild-type protein (FLwt) and that this effect is reduced in the presence of the mutant protein (FLmu).

FIG. 4

EMSA (Electrophoretic-Mobility-Shift-Assay) performed on cytoplasmic (C) and nuclear (N) extracts obtained from parental cells (P) and from cells overexpressing wild-type and mutant huntingtin (FLwt and FLmu) incubated with the NRSE DNA sequence which has been radioactively labelled. The complexes (indicated with number 1 and 2), which are composed by this sequence and by the factors which bind to it are separated by electrophoresis on a polyacrilamide gel in non denaturing conditions and then revealed by autoradiography. This experiment shows that complexes 1 and 2 are well evident in the cytoplasm (C) of cells overexpressing wild-type huntingtin. The opposite occurs in the nucleus. On the contrary, complexes 1 and 2 are poorly visible in the cytoplasm (C) when the mutant protein is overexpressed, but they are well evident in the nucleus (N). Co+ is the positive control of the experiments represented by nuclear extracts from Hela cells incubated in the presence of the radioactive NRSE sequence; as observed, complexes 1 and 2 are well evident. Co− is instead the negative control in which the radioactive NRSE element is incubated in the absence of the nuclear lysate. In this case complexes 1 and 2 are absent because there is no binding of transcription factors to the NRSE sequence.

FIG. 5

Western Blotting analyses of cytoplasmic (C) and nuclear (N) distribution of transcription factors binding to the NRSE sequence.

It is possible to observe from the figure that, while HDAC1, mSin3a and coREST distribution in the cytoplasm and in the nucleus are similar between cells expressing wild-type (FLwt) and mutant huntingtin (FLmu) REST transcription factor accumulates in the cytoplasm (C) of FLwt cells while it is less present in the nucleus (N) of the same cells. On the contrary, in FLmu cells the presence of REST in the cytoplasm (C) is reduced while an accumulus is observed in the nuclear fraction (N). The evaluation of tubulin concentration in cytoplasmic extracts and histone H1 in nuclear extracts constitutes an important quantitative indicator in the analysis of the transcription factors distribution previously described.

### DETAILED DESCRIPTION OF THE INVENTION

The present invention relates to a method for the selection of molecules useful for the treatment of Huntington's Disease characterised by the use of the NRSE sequence as a molecular target of said molecules.

The method allows to identify those molecules that, by blocking the silencing activity of the NRSE target sequence, reproduce the neurotrophic and neuro-protective effect of huntingtin; the evidence of such inhibition, which is on the basis of the physiologic neuronal protective mechanism lost in Huntington's Disease, constitutes a drug screening methods, with a high discriminanting power, for the isolation of molecules useful to prevent and/or treat Huntington's Disease.

In general, the method is based on the incubation of the candidate compound with cells containing the NRSE sequence to evaluate if the repressive effect on gene transcription is inhibited.

To reveal and quantify the activity of the NRSE element and its inhibition it is possible, to use cells which are engineered in order to stably contain the NRSE sequence upstream of a reporter gene so that the activity of the reporter gene is regulated by the NRSE sequence. The reporter gene contains a DNA sequence modified so that its expression, which is an index of the activity of the NRSE sequence, can be measured by chemical, physical or chemical-physical analyses. Any reporter gene may be used in this invention: non-limiting examples are the CAT (chloramphenicol acetyle transferase) gene, the LUC gene (luciferase) or the GFP (Green Fluorescent Protein) gene; in particular, the last two reporter genes present the advantage of allowing an automated quantitative measurement without the use of radioactive reagents.

The present invention uses, as a substrate for carrying out the method, a cell system stably containing in its genome the NRSE sequence, which controls a reporter gene located downstream, The cell system is produced by transfecting the cells with a suitable vector which comprises the NRSE sequence upstream of the reporter gene of choice; cells are transfected to stably express the NRSE sequence (target of huntingtin) and the activity of this sequence is measured by revealing gene reporter activity.

Preferably, the cells forming the cell system used in the assay are preferably neural cells and more preferably neural striatal cells.

The vector used for the transfection which contains the NRSE sequence upstream of the reporter gene (useful to produce the cellular system mentioned above) is a further object of the invention. The NRSE-TK-LUC vector, produced by the applicants and described in the experimental methods, is an example of this vector.

The execution of this screening method for the isolation of compounds able to treat prevent Huntington's Disease requires that the candidate compound is added to the cellular system described above appropriately pre-propagated.

As a non-limiting example of the present method, the cells are propagated at a temperature comprised between 30° C. and 40° C.; the atmosphere to which they are exposed contains 5% CO₂; the culture medium is represented by Dulbecco Modified Eagle Medium containing Sodium Piruvate 0,11 g/l, L-glutamine 2 mM, Penicilline/Streptomycine 120 μg/ml supplemented with Fetal Calf Serum 10% final concentration. When propagation is finished, for example after 24 hours from the seeding of the cells, the compound to be tested is added to the culture medium at a concentration usually ranging between 1 nM and 10 μM. The activity of the NRSE element is evaluated at different time points, starting at 6 hours after the administration of the compound until 48-72 hours, and it is quantified by the use of the specific detection reaction for the reporter gene used in the assay; preferably the detection assay is carried out on cellular lysates obtained from the treated cells, as described in the literature and reported in the experimental section. In parallel to the test with the candidate compound, a control test is used, in which the same system is used without incubation with the selected compound. The two values obtained are compared.

An increased gene reporter activity in the treated sample compared to control indicates that the candidate compound inhibits the activity of the NRSE element, therefore mimicking wild-type huntingtin function. This compound is potentially useful as a drug to prevent and/or treat Huntington's Disease. An unchanged activity compared to control indicates that the candidate compound is devoid of an inhibitory effect on the NRSE sequence and will not be further considered.

A decreased activity compared to control indicates that the candidate compound is able to increase the silencing activity of the NRSE sequence which results in a toxic effect: in this case the method is useful for example, in the determination of the toxic effect (pro-choreic effect) of possible polluting agents; these determinations may be of interest for the control of environmental health and more generally, for the better understanding of the pathogenesis of the disease.

For the candidate compounds that, by the use of the method describe above, have shown a potential therapeutical interest, it is possible to repeat the test under more stringent conditions with the final aim to evaluate the intensity and persistence of the inhibitory effect on the NRSE sequence.

To this purpose the candidate compound is further analyzed as described above, but in this case using a cellular system in which the NRSE sequence and the reporter gene are stably expressed in cells engeneered to overexpress mutant huntingtin.

For the purpose of the present invention, the term “mutant huntingtin” means any form of huntingtin which is not able to reproduce the physiological effect of wild-type huntingtin, which is the inhibition of the NRSE sequence.

In a cellular system that overexpress mutant huntingtin, the pro-chroreic effect (due to the lack of the physiological, neuroprotective action of huntingtin) is considerably present.

Examples of cells over expressing huntingtin in its mutant form are those containing the expanded pathological polyglutamine tract; these cells and the procedure to obtain them have been previously described by the applicants (Rigamonti et al, J. Nevrosci, 20(10), 2000, pp 3705-3713, incorporated werein by reference). A persistent NRSE inhibition by the candidate compound, revealed by an increase in the gene reporter activity even in these stringent conditions, is a further element that confirms its usefulness to treat and/or prevent the disease.

Further studies performed by the Applicants have shown that wild-type huntingtin's action on the NRSE element is based on the retaining in the cytoplasm of a transcription factor which is necessary to evoke the silencing activity of the NRSE sequence.

This factor is called REST (Restriction Element Silencer Factor, Palm et al J. Neurosci, 18(4), 1998, pp 1280-1296) and it is a protein able to bind directly the NRSE element thanks to the presence of 9 structural specific motives allowing its interaction with the DNA of said sequence.

The Applicants have demontrated that in physiologic conditions (in the presence of the neuroprotective activity of wild-type huntingtin) REST transcription factor is maintained in the cytoplasm and delivered to the nucleus' only in small amounts. In pathological conditions (in the absence of the protective function of wild-type huntingtin and thus in the presence of mutant huntingtin) REST transcription factor is no longer retained and it is delivered to the nucleus. Once in the nucleus, it activates the silencing action of the NRSE sequence thus reducing the production of BDNF neurotrophic factor (FIG. 5).

Therefore, in a further step of this invention, the compounds, to be tested for example those previously identified as able to inhibit the activity of the NRSE sequence may be undergo an analysis to evaluate their ability to control the cytoplasmic and nuclear distribution of REST.

High cytoplasmic levels (and/or low nuclear levels) of REST are indicative of a neurotrophic activity which is similar to the activity of wild-type huntingtin and therefore of potential therapeutical efficacy; unchanged values indicate inactive compounds; low cytoplasmic levels (and/or high nuclear levels) are instead indicator of pro-choreic neurotoxic activity.

Cell cultures, preferably of neural cells and more preferably of striatal cells are used as substrates for the detection and quantification of cytoplasmic and nuclear distribution of the REST factor; the cells may be parental cells or cells that have been engineered to express mutant huntingtin. They are incubated with the compound to be tested. At the end of the incubation the cytoplasmic and/or nuclear proteic lysates are isolated and these are then used to quantify (by comparison with the basal levels of non-treated cells) the variation of the REST levels resulting from the treatment with the candidate compound. The REST levels may be analysed with known techniques, for example, by Western Blotting: the amount of REST protein in the cytoplasm and in the nucleus of these cells is evaluated by the use of monoclonal antibodies, molecules able to recognize specifically this protein.

Alternatively to the Western Blotting technique, the Applicants have developed a second system which allows do detect in a simpler but equally effective way, the nuclear and cytoplasmic distribution of the REST protein The Applicants have developed an expression construct bearing the REST protein cDNA sequence fused to the fluorescent marker called Green Fluorescent Protein (GFP). The cDNA sequence for REST, located between the restriction sites NcoI and XbaI, 3029 bp long (NCBI accession number AB024496), has been cloned in the plEGFPN1 vector (Clontech). The vector obtained containing the sequence encoding for REST-GFP fusion protein is stably transfected in the above said cells. These cells are grown as previously described. After an appropriate growth period, for example 24 hours after seeding, the candidate compounds are added to the culture medium.

Nuclear and cytoplasmic distribution of REST protein is evaluated by confocal microscopy on the cultured cells. An intense green fluorescent signal observed in the cytoplasm (due to the GFP protein which, when stimulated with a specific wavelength emits a green light), compared to untreated control cells, indicates that the compound used in the assay is able to mimick wild-type huntingtin function in retaining the REST protein in the cytoplasm. Said compound is therefore potentially useful in the cure of the pathology because it is able to inhibit NRSE activity and to retain the REST transcription factor in the cytoplasm. The detection of a fluorescent signal similar to the control is instead indication of a lack of effect of the administered compound, while an accumulation of green fluorescent signal in the nucleus is indication of a neurotoxic or pro-choreic action.

The evaluation of the REST levels may also be used as a control in the NRSE activity inhibition test previously described. In such a case, in addition to the two tests carried out in the presence and absence of the candidate compounds which have been described above, a third test is added wherein a compound with a known action on the NRSE sequence (for example wild-type huntingtin) is added to the cell system; an increase in the reporter gene activity indicates that the cell system used works correctly.

The compounds with a clear anti-choreic activity, detected by the use of the methods described herein, may undergo the following course of pharmacological experimentation. The obtained methods are useful to isolate, by the use of a simple test that excludes animal sacrifice, a high number of candidate compounds (for example series of homologue drugs obtained by combinatorial synthesis); the further experimental course will be limited to the only compounds that were already active in the previous test. Therefore, the invention described here gives the opportunity of a molecular assay of simple execution that can be routinely applied to isolate an active compound among a large number of molecules.

As described above, the Applicants showed that the inhibition of the NRSE sequence mediated by huntingtin is the neuro protective mechanism that is lost in Huntington's Disease. This is caused by the accumulation of the REST protein in the nucleus (REST is the transcription factor that by binding directly to the NRSE sequence is able to activate the silencer activity of this element). Therefore, this invention comprises the use of compounds which inhibit the NRSE sequence and/or which block the nuclear accumulation of the REST factor for the preparation of a medicament useful to prevent and/or treat Huntington's Disease.

The invention will be now explained by the following not restrictive examples.

1. Experimental Section

Experimental Model

An immortalized cell line obtained from cells at embryonic day 14 and known with the name of ST14A cells is available to the Applicants (Cattaneo et al, Brain Res. Dev. Brain Res., 83(2) 1994, p. 197-208; Cattaneo & Conti, J. Neurosci Res., 53(2), 1998, 223-34).

Engineered sub-clones overexpressing full-length wild-type or mutant huntingtin were previously derived from these cells (Rigamonti et al., 2000, see above).

In particular, besides the ST14A parental cells, the sub-clones used in the study are the following:

FLwt (“Full Length wild-type huntingtin”): ST14A cells engineered with the cDNA encoding for the human full-length wild-type huntingtin bearing 23 CAG repeats.

FLmu (Full Length mutant huntingtin”): ST14A cells engeneered with the cDNA encoding for the human full-length mutant huntingtin bearing 82 CAG repeats.

The lines described above are maintained and grown in DMEM medium supplemented with 10% FCS (Fetal Calf Serum), 0.11 g/l Na Piruvate, 2 mM L-glutamine and 120 μg/ml Pen-Strep.

The cells are grown in plates at 33° C. degrees in the presence of 5% CO₂ and propagated when they are 90% confluent.

Previous results obtained by the Applicants have shown that the engineered sub-clones stably express exogenous huntingtin (Rigamonti et al., 2000 see above) and are a good in vitro model for the study of wild-type huntingtin function and for the study of the biochemical and molecular mechanisms at the basis of Huntington's Disease pathogenesis.

2. Methods

2.1 Stable Transfection and Assays to Reveal the Activity of Luciferase Reporter Gene

After 24 hours from seeding, the cells are transfected with 14 μg of NRSE-TK-LUC vector. Lipofectamine is used for transfection experiments following the protocol suggested by the manufacturer (Invitrogen/Life Technologies). Cells are then grown for 15 days in the culture medium supplemented with puromicin (a selective agent specific for the NRSE-TK-LUC vector) with the aim to select clones (a group of cells derived from a single cell) that stably express NRSE-TK-LUC vector, which means that the vector is inserted in the genome of the transfected cells and replicates together with it at every cell division.

A preliminary test for luciferase activity will be performed on the selected clones with the aim of controlling the insertion of the construct in loci of the cellular genome able to permit its expression.

The cells stably expressing the NRSE-TK-LUC are plated at 85% confluence in 2 cm² wells (1-1,5×10⁵ cells/well) in the presence of the tested compound. The cell culture medium is completely removed 48 hours after the administration of the compound. At this point, 120 μl of lysis buffer (Luciferase Lysis Buffer 5×: 40 mM tricine pH 7.8, 50 mM NaCl, 2 mM EDTA, 1 mM MgSO₄, 1 mM DTT, 1% Triton X100) are added to each well, and, after 5 minutes of incubation at room temperature, the lysates are transferred in ice-cold tubes. At the meantime, the Luciferase Assay Reagent 1× (20 mM Tricina; 0.1 mM EDTA; 1.07 mM (MgCO₃)₄Mg(OH)₂.5H₂O; 2.67 mM MgSO₄.7 H₂O; 33.3 mM DTT) is prepared to which 270 μM Coenzyme A, 530 μM ATP and 470 μM luciferine have been added. 100 μl of Luciferase Assay Reagent 1× are added to 20 μl of lysates and the luciferase activity in the presence of luciferin, is revealed by the use of a luminometer.

2.2 Stable Transfection, Cellular Lysate Preparation and Assays for Cat Reporter Gene Activity

The cells described above seeded in 100 mm plates are cotransfected at confluence with 3,5 μg of plasmid DNA represented by the NRSE-TK-LUC vector and 0,5 μg of plasmid encoding β-galactosidase (an internal control for transfection efficiency). In the first construct the inserted sequence, containing the NRSE element, is a 91 base pair sequence ranging from nucleotide 1873 and nucleotide 1964 of the rat BDNF gene (Timmusk et al., Neuron, 10, 1993 pp 475-489). This sequence has been inserted in the pBLCAT 2 vector (Luckow and Shutz, Nucl Acids Res. 15, 1987 p 5490) at the restriction site SalI. The vector containing β-galactosidase is pSV-βGalactosidase (Promega). The transfection method is based on the use of Lipofectamine Plus and it is performed following the instructions of the manufacturer (Invitrogen/Life Technologies).

48 hours after the addition of the compound to be tested to the culture medium, cells are collected, pelletted and lysed after resuspension in 150 μl of lysis buffer (15 mM Tris-HCl pH 7.5, 60 mM KCl, 15 mM NaCl, 2 mM EDTA pH 8, 1 mM DTT e 0.4 mM PMSF). The lysis protocol is based on five cycles of freeze-thawing (−80° C./37° C.) 5 minutes long. The lysates are then centrifuged at 14000 rpm for 5 minutes at 4° C.: the supernatant are recovered and represent the protein lysate. The protein content is evaluated by Bradford Assay. The β-galactosidase assay is performed on a aliquote of the samples, as described in Maniatis T., Molecular Cloning: A Laboratory Manual. Cold Spring Harbor Laboratory Press, NY, Vol. 12, 3 (1989).

The CAT reporter assay is performed as follows:
The protein lysates are incubated 15 minutes at 65° C. to inactivate deacetylation enzymes.
the samples are centrifuged at room temperature at 14000 rpm for 15 minutes.

The supernatants are collected and the CAT assay is set up: 90 μl of protein lysates corresponding to 100 μg of proteins are incubated at 37° C. for 2 hours with 10 μl of MASTERMIX (0,5 μl AcetylCoa 70 mg/ml, 0,1 μC ¹⁴C Chloramphenicol and water to the final volume). The acetylated forms of chloramphenicol are extracted by adding 500 μl Etylacetate to each sample. The samples are mixed with the vortex, centrifuged and then the reaction products are collected by drying the upper phase with a nitrogen flux. After resuspension with 30 μl Etylacetate the samples are then loaded on silica gel and separated by TLC in a mixture containing chloroform and methanol in a 95:5 ratio. The following steps involve drying and exposure for 3-5 days at −80° C. to an autoradiography film Kodak Biomax.
For the final evaluation of CAT activity, the reaction products are recovered from the TLC plates and the radioactive signal is quantified by the use of a β-counter. The obtained results are normalized on transfection efficiency.
   2.3 Analysis of Cytoplasmic and Nuclear Distribution of Rest Protein.
   (I) Western Blotting for the Identification of Rest Protein

Cells grown in 100 mm plates are deprived of culture medium, washed with 2 ml of PBS 1× and datached from the plate with 2 ml of 2 mM PBS 1×-EDTA pH8 after slow agitation. Following centrifugation for 5 minutes, 1050 rpm, at room temperature, the obtained cell pellets are immediately frozen on dry ice and stored at −80° C.

Cytoplasmic and nuclear lysates preparation is performed by the following steps:
the pellet of cells is resuspended in 100 μl of buffer A (50 mM NaCl, 10 mM NaHepes pH 7.6, 500 mM sucrose, 1 mM EDTA, 0.5 mM spermidine, 0.15 mM spermine, 0.2%. Triton-X100) supplemented with protease inhibitors cocktail (Sigma) and PMSF (Phenyl Methyl Sulphonyl Fluoride). The resuspended cells are incubated 2 minutes on ice. The following step is centrifugation at 4° C. for 2 minutes at 6000 rpm. The recovered supernatant represents the cytoplasmic lysate.
100 μl of buffer B (50 mM NaCl, 10 mM NaHepes pH 8, 25% glycerin, 0.1 mM EDTA, 0.5 mM spermidine, 0.15 mM spermine) are then added to the cell pellet and removed immediately. This step is repeated three times. The samples are centrifuged for 30 seconds at 6000 rpm at 4° C. and the supernatant is removed.
The pellet is then resuspended with 20 μl of buffer C (350 mM NaCl, 10 mM NaHepes pH 8, 25% glycerine, 0.1 mM EDTA, 0.5 mM spermidine, 0.15 mM spermine). The samples are incubated for 20 minutes on ice. Thereafter, they are centrifuged for 15 minutes at 4° C. at 14000 rpm: the recovered supernatant represents the nuclear lysate.
Cytoplasmic and nuclear lysates are dialyzed under stirring for 3 hrs at 4° C. against a solution composed by 20 mM NaHepes pH 7.9, 20% glycerin, 200 mM NaCl, 0.2 mM EDTA, 0.2 mM PMSF and 0.5 mM DTT. Protein content is then evaluated by Bradford reagent. 100 μg of cytoplasmic and nuclear protein lysates are separated by SDS-PAGE on a 10% polyacrilamide gel. Electrophoresis is performed at room temperature at costant voltage (150 V) with a buffer composed by 0.025M Tris-HCl, 0.2 M Glycine and 3.4 mM SDS. Protein transfer from the gel to a nitrocellulose membrane is performed at 4° C. with costant amperage (250 mA); the membrane is pre-treated for few minutes in transfer buffer (2 M glycine, 0.25 M Tris-HCl, 20% methanol and 0.2% SDS). Then, the membrane is saturated over-night with 5% no fat milk prepared in TBS-T 1× (TRIS 20 mM, NaCl 500 mM, Tween 20 0.1%). The day after the membrane is incubated with antiREST monoclonal primary antibody (gently provided by David Anderson, Caltech, Calif.) for 2 hours at room temperature. Following repeated washes with TBS-T 1×, the membrane is incubated for one hour with of the appropriate secondary antibody diluted 1:10000 in 5% no fat milk in TBS-T 1×. After repeated washes the autoradiography signal is revealed by chemioluminescent analysis (ECL Amersham). Tubulin and H1 proteins are used as loading control.
   II) Stable Transfection of REST-GFP Expression Construct and Confocal Microscopy Analyses of Cytoplasmic and Nuclear Distribution of the Fusion Protein.

The cDNA sequence encoding for REST protein was cloned upstream of the Green Fluorescent Protein (GFP) in pLEGFPN1 vector (Clontech). This vector is stably expressed in parental striatal cells and in cells over expressing mutant huntingtin previously engineered with a reporter gene fused downstream of the NRSE element.

The stable cell clones, whose production is described above, are grown and then treated with the compounds able to inhibit the NRSE silencer activity at doses and time courses as previously identified. Then, the cytoplasmic and nuclear distribution of REST transcription factor is observed by confocal microscopy at the right wave length to see the green fluorescent signal produced by GFP protein.

3. Results

The results here described identify, in the BDNF gene promoter, the specific DNA element on which wild-type huntingtin exerts its action. This is the NRSE element, a sequence that, once activated, blocks the expression of genes that contain these sequence.

Further, the mechanism by which wild-type huntingtin acts on the NRSE element is explained and this is the reduced availability of REST transcription factor to the nucleus due to the ability of wild-type huntingtin to maintain REST in the cytoplasm. The reduction of this protein in the nucleus, which is crucial for the activation of the NRSE silencer sequence, causes the inhibition of its silencing activity, allowing BDNF expression. The results obtained indicate the availability of a molecular target that can be used to isolate drugs active in the pathology, able to mimick wild-type huntingtin function on the NRSE element thus increasing BDNF transcription.

(i) Identification of NRSE Element as a Target of Wild-Type Huntingtin Action

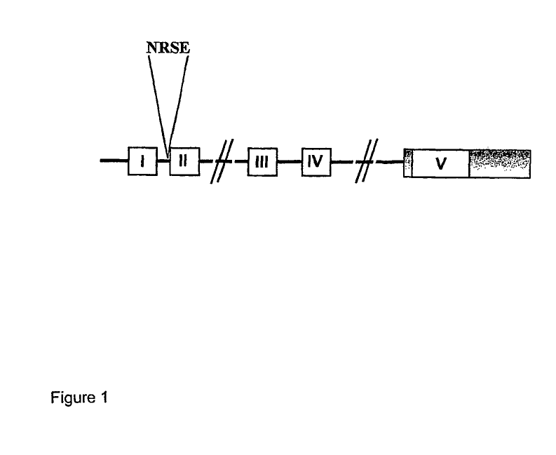

With the aim to identify the specific portion in the 1100 base pairs sequence (encompassing promoter II and a part of the upstream regulatory sequence, FIG. 1) on which wild-type huntingtin exerts its action, the Applicants have used different DNA fragments, obtained from deletions of the 1100 bp sequence, which were inserted in plasmid vectors to drive a specific sequence represented by the CAT gene (chloramphenicol acetyle transferase), whose expression can be easily identified by a chemical assay.

Once this construct is inserted in the cellular system, the activity of the CAT enzyme and that of the regulatory sequence upstream of it, is evaluated as index of the amount of chloramphenicol substrate that it is converted in the corresponding final reaction products. These products are isolated by thin layer cromatography and then quantified.

The constructs used in the assay are the following:
BDNF II 1,1 CAT: it contains a 1,1 Kb sequence between the restrictions sites Hind III and SacI which comprises a portion of promoter II and a portion of the upstream region containing a NRSE element.
BDNF II 0,3 CAT: it contains a sequence of 300 bp comprising the same portion of promoter II which is present in the vector described above. A deletion in 5′ end is present compared to the previous construct, but the NRSE sequence is maintained.
BDNF II 0,3. MUT CAT: it contains a sequence of 300 bp comprising same portion of BDNF. II promoter present in the previous vector, but the NRSE element is mutated.

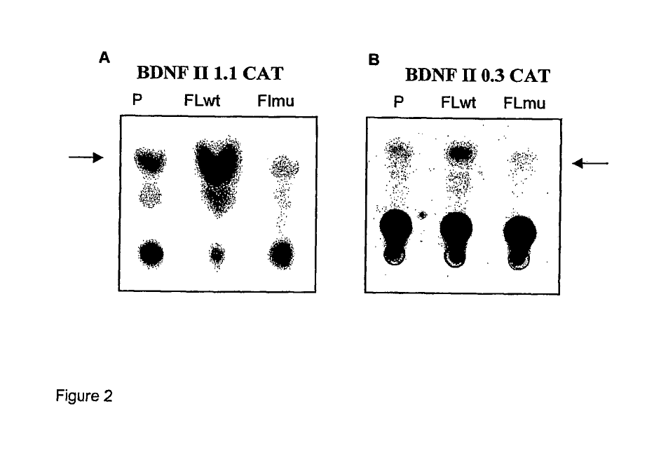

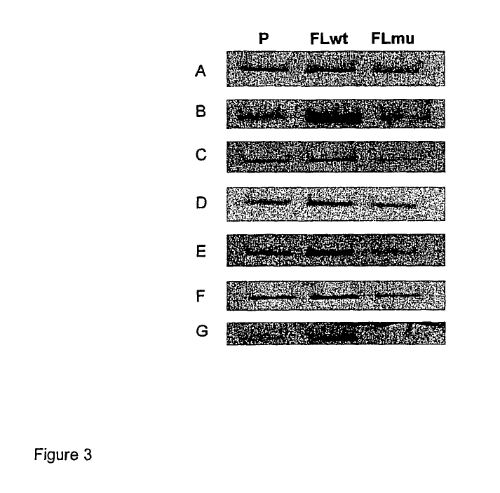

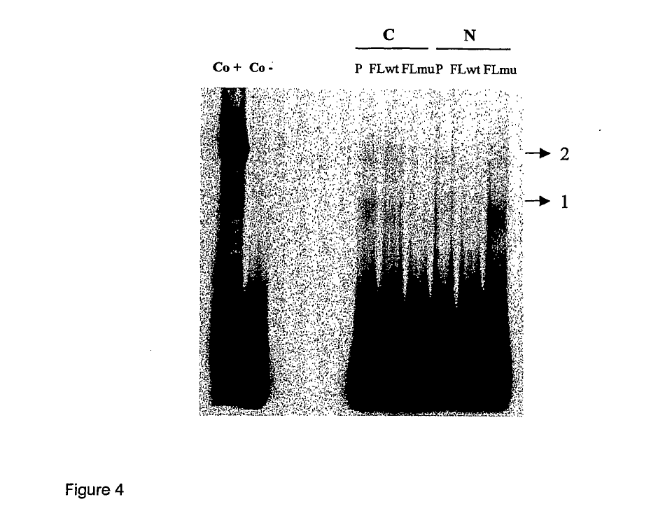

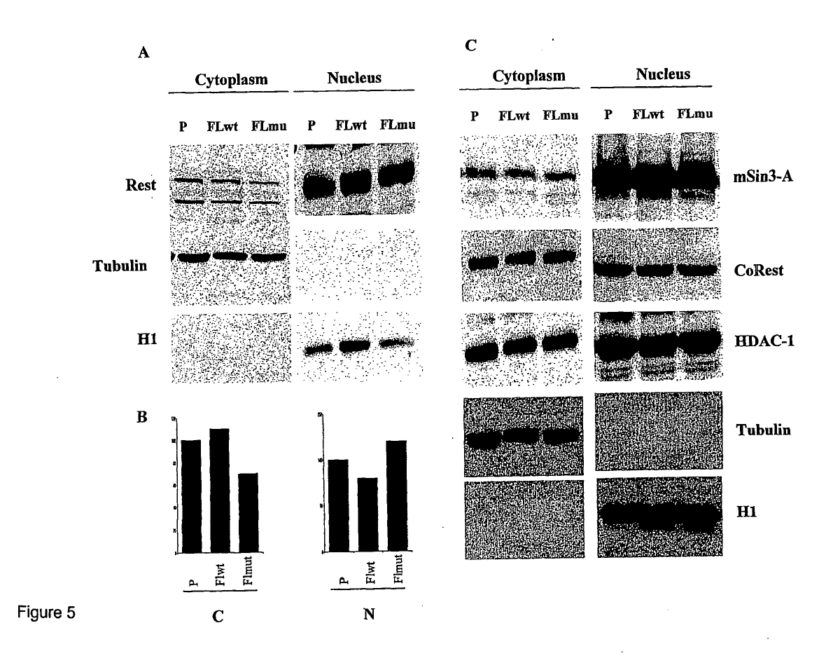

The BDNF 111,1 CAT and BDNF II 0,3 CAT vectors independently were transiently transfected in ST14A parental cells and in ST14A cells engineered to overexpress wild-type and mutant huntingtin (Rigamonti et al., 2000, see above). CAT activity was evaluated after transfection: CAT activity is increased in the presence of overexpressed wild-type huntingtin compared to control, on the contrary this activity is reduced in the presence of the mutant protein compared to parental cells (FIG. 2, panels A and B and Table I).

TABLE I

| | BDNF II 1.1 CAT | BDNF II 0.3 CAT |
|---|---|---|
| P | 1851.5 +/− 450 | 4875 +/− 245 |
| FLwt | 18062 +/− 975** | 8549 +/− 315** |
| FImu | 494.5 +/− 280* | 2845 +/− 177* |

Tabel I shows the quantitative analysis of the acetylated products (acetylated chloramphenicol indicated with an arrow in FIG. 2) shown in the experiments of panel A and B. CAT activity data are expressed in cpm (count for minute/micrograms of protein lysate/transfection efficiency) and show means with standard deviation of three different experiments *P<0.50.01 versus P. ANOVA Test.

For quantification the areas corresponding to the acetylated product have been isolated from the TLC plate and counted using scintillation liquid.

The same assay was repeated by the use of plasmid BDNF II 0,3 MUT CAT in parental cells (P) or cells over expressing wild-type (FLwt) or mutated (FLmu) hintingtin.

The BDNF II 0.3 NUT CAT vector contains the 300 base pairs sequence of promoter II shown in FIG. 2, but with a NRSE element which has been mutated as follows: 5′TCCgggacGCAgaTTttACAGAGCCAGCGGATTTGTttGAcaTGGTAGTA CTT3 (the bases that have been mutated are indicated in small caps). The results obtained are shown in table II, in which data are expressed in cpm (count for minute/micrograns of protein lysate/transfection efficiency) and show means with standard deviation of three different experiments.

TABLE II

| | BDNF II 0.3 MUT CAT |
|---|---|
| P | 4875 +/− 245 |
| FLwt | 5214 +/− 915 |
| FLmu | 3287 +/− 875 |

The table shows that in the presence of the NRSE element the CAT activity in FLwt and FLmu cells does not vary significatively compared to the control represented by the parental cells; thus no modulation of CAT activity compared to the control is observed both in the presence of wild-type and of mutated hungtintin.

This means that the applicants found the minimum DNA sequence which is necessary for wild-type huntingtin to exert its action on BDNF gene transcription; this is the NRSE sequence.

(ii) Wild-Type Huntingtin Stimulates the Expression of Other Genes Regulated by the NRSE element.

As previously described, the NRSE element is a sequence that, once activated by specific proteins, blocks the expression of the genes controlled by it.

The NRSE sequence is localized in the promoter region of many neuronal genes responsable for the survival and maintainance of the neuronal phenotype. They include neurotransmitter receptors, ion channels, enzymes which control neurotrasmitter synthesis, neuropeptides, molecules for cellular adhesion, synaptic vescicle proteins and cytoskeletal components.

On the basis of the original results here reported, that demonstrate that huntingtin modulation of BDNF gene expression is due to an activity on the NRSE sequence, the Applicants have tried to identify if the expression of other genes regulated by the NRSE element could be influenced by wild-type huntingtin, as observed for the BDNF gene.

The Applicants produced primers for some of these genes and performed semiquantitative radioactive RT-PCR experiments to verify their expression levels in cells expressing wild-type and mutant huntingtin. As shown in FIG. 3 it is possible to see that in the presence of wild-type huntingtin their expression is increased, compared to control cells while their expression strongly decrease in the presence of the mutant protein. This highlights that, besides the BDNF gene, wild-type huntingtin is able to regulate a group of neuronal genes, whose expression is controlled by the NRSE element.

(iii) Production of a Cellular System which Express the NRSE Element, the Target of Wild-Type Huntingtin Action.

In order to make use the of regulatory mechanism of wild-type huntingtin in the identification of drugs active in the disease, the Applicants have produced the following vector:
NRSE-TK-LUC: it has been obtained from the NRSE-TK-CAT (obtained by the insertion of the NRSE sequence in pBLCAT2 vector upstream of the TK promoter) by substitution of the CAT reporter gene with the gene encoding for luciferase (isolated from pGL3luciferase, Promega)

This construct was transiently expressed in the cellular system indicated above and it was observed that in wild-type huntingtin overexpressing cells luciferase activity was increased; on the contrary in the presence of the mutant protein luciferase activity is at a level that is lower than control.

(iv) Huntingtin Inhibits NRSE Activity by Sequestering in the Cytoplasm Rest Transcription Factor, a Crucial Factor for the Silencing Activity of the NRSE Element.

The Applicants have analysed throughly the mechanisms by which huntingtin acts on the NRSE element and they have identified that the wild-type protein regulates the nuclear import of NRSE binding transcription factors (FIG. 4). This ability is lost in the presence of the mutation with the consequent nuclear accumulation of transcription factors. This causes an increase in the silencer activity of the NRSE element.

Evidence of this was obtained by EMSA assay (Electrophoretic-Mobility-Shift-Assay) shown in FIG. 4.

By this assay the Applicants found that:
In cells overexpressing wild-type hungtintin, the complexes, formed between the NRSE sequence and the factors able to bind and activate its silencing activity are preferentially present at a cytoplasmic level. The nuclear signal is very weak.
In cells overexpressing the mutated protein, the above complexes are more evident in the nucleus and tend to disappear in the cytoplasm.

Further Western Blotting experiments, which were focused on the identification of the cytoplasmic and nuclear distribution of the transcription factors able to bind to the NRSE, and to activate it demonstrate that no differences are revealed compared to control (FIG. 5A) between the cytoplasmic and nuclear distribution of mSin3a, HDAC1, coREST transcription factors in the presence of wild-type and mutant huntingtin. It has instead been observed that REST transcription factor, the crucial protein of this complex since it is able to bind directly the NRSE sequence and to recruit the other factors in the transcription machinery, remains in the cytoplasm of wild-type huntingtin cells, while it is reduced in the cytoplasm of cells with mutant huntingtin and accumulate in the nucleus (FIG. 5B).

This clearly shows that the BDNF stimulating action of wild-type huntingtin is due to the cytoplasmic sequestration of REST transcription factor which is necessary to activate the NRSE silencer sequence.

1. A method for the selection of molecules active in the prevention and/or treatment of Huntington's Disease wherein the ability of said molecules to inhibit the activity of the NRSE element is evaluated.

2. A method according to claim 1 comprising the following steps:
(a) incubation of said molecules in presence of a cell system, stably transfected with the NRSE sequence inserted upstream of a reporter gene,
(b) evaluation of inhibition of the NRSE sequence activity by measurement of gene reporter activity.

3. A method according to claim 2, wherein said reporter gene is selected from the group consisting of the chloramphenicol acetyle transferase gene, the luciferase gene and the green fluorescent protein gene.

4. A method according to claim 2, wherein said cells are cells expressing mutated huntingtin.

5. A method according to claim 2, further comprising the evaluation of the amounts of REST factor in the cytoplasm and/or nucleus of said cells.

6. A cellular system suitable to perform the method described in claim 1 wherein it consists of cells that are engineered to stably contain the NRSE sequence inserted upstream of a reporter gene.

7. A cellular system according to claim 6 wherein the reporter gene is selected from the group consisting of the chloramphenicol acetyle transferase gene, the luciferase gene and the green fluorescent protein gene.

8. A cellular system according to claim 6, whose cells are neuronal cells.

9. A cellular system according to claim 8, whose cells are striatal cells.

10. A cellular system according to claim 6 whose cells are either parental cells or cells expressing huntingtin.

11. A process for the production of the cellular system described in claim 6 that comprises:
(a) the obtainment of a vector with the NRSE sequence inserted upstream of a reporter gene and
(b) transfection of the cells of this system with said vector.

12. A process according to claim 11, wherein said vector is the NRSE-TK-LUC construct.

13. A transfection vector suitable to be used in a process according to claim 11, whose sequence comprises the NRSE sequence inserted upstream of a reporter gene.

14. A transfection vector according to claim 13, wherein the reporter gene is selected from the group consisting of the chloramphenicol acetyle transferase gene, the luciferase gene and the green fluorescent protein gene.

15. A vector according to claim 13 which is the NRSE-TK-LUC construct.

16. A method for the selection of molecules able to act in the prevention and/or treatment of Huntington's disease comprising the following steps:
(a) incubating said molecules with a cellular system
(b) evaluating the decrease of REST transcription factor in the cytoplasm and/or its increase in the nucleus of said cells.

17. Therapeutic method for the prevention and/or treatment of Huntington's disease comprising administering to a subject in need thereof a therapeutically effective amount of a NRSE inhibitor.

18. A NRSE inhibitor compound, selected by the method described in claim 1.

19. A NRSE inhibitor compound, selected by the method described in claim 16.
